(19) United States
(12) Patent Application Publication
(10) Pub. No.: US 2021/0339037 A1
(43) Pub. Date: Nov. 4, 2021

(54) PHYSICAL TARGETED HYPERTHERMIA SYSTEM USED FOR TUMOR THERAPY AND CONTROL METHOD THEREOF

(71) Applicant: Yinghui Sun, Shanghai (CN)

(72) Inventor: Yinghui Sun, Shanghai (CN)

(21) Appl. No.: 16/931,849

(22) Filed: Jul. 17, 2020

(30) Foreign Application Priority Data
Apr. 29, 2020 (CN) 202010359015.2

(51) Int. Cl. A61N 5/02; A61N 5/04

(52) U.S. Cl. CPC A61N 5/025; A61B 2018/1823; A61N 5/04

(57) ABSTRACT

This application provides a physical targeted hyperthermia system used for tumor therapy and a control method thereof. The system mainly includes a hyperthermia apparatus body, a microwave radiation device, a microwave receiving device, a microwave imaging unit, a microwave temperature-measuring unit, a hyperthermia solution generating unit, a hyperthermia solution executing unit, and a hyperthermia control device. The hyperthermia apparatus body is used to provide stable supporting surface for a patient and to provide an mounting sites for related function devices, the microwave radiation device is used to generate a microwave with a set wavelength and a set frequency radiating toward a set direction, the microwave receiving device and the microwave imaging unit are used to accurately position the tumor tissue, the hyperthermia solution generating unit is used to generate hyperthermia parameter information, and the hyperthermia solution executing unit is used to generate corresponding hyperthermia solution execution information.

### CROSS REFERENCE TO RELATED APPLICATIONS

This application claims priority to Chinese Patent Application No. 202010359015.2, filed on Apr. 29, 2020. The entirety of the above-mentioned patent application is hereby incorporated by reference herein and made a part of this specification.

### TECHNICAL FIELD

This application relates to the technical field of medical equipment, and more particularly, to a physical targeted hyperthermia system used for tumor therapy and a control method thereof.

### BACKGROUND

Currently, radiotherapy, chemotherapy, and surgical therapy are commonly used methods for tumor therapy. Radiotherapy refers to a local therapy method in which a radioactive ray is used to treat a tumor. For example, a, (3, and y rays generated by radioisotopes and x-rays, electron rays, proton beams, and other particle beams generated by various x-ray therapy machines or accelerators radiate or bombard tumor cells, so that tumor cells in a target area are eliminated. Chemotherapy, short for chemical drug therapy, is a method in which a chemotherapy drug is used to kill cancer cells so as to achieve the purpose of therapy. Surgical therapy, as the term suggests, is a method in which surgical resection is used to remove tumor tissue from normal tissue of the human body so as to achieve the purpose of therapy.

Chemotherapy, radiotherapy, and surgical therapy are important methods for treating malignant tumors, having the advantages of killing tumor cells and inhibiting their growth and expansion. It also has obvious side effect, that is, it will damage normal cells or immune cells of the human body during the therapy, resulting in decreased immune function of the patient, myelosuppression, suffering from radiation pneumonia and so on.

How to kill tumor cells without damaging normal cells or tissue in human, that is, achieving accurate killing of tumor cells, is a difficult problem to be overcome.

### BRIEF SUMMARY

Regarding the side effect of damaging normal cells or immune cells of the human body during the therapy, this application provides a physical targeted hyperthermia system used for tumor therapy and a control method for the physical targeted hyperthermia system used for tumor therapy, and the specific solution is as follow.

A physical targeted hyperthermia system used for tumor therapy, includes:

a hyperthermia apparatus body configured to provide at least one stable supporting surface for a patient during hyperthermia and provide mounting positions for individual system function modules;

a microwave radiation device in connection with a microwave source and configured to generate a microwave with a set wavelength and a set frequency radiating toward a set direction according to a control signal;

a microwave receiving device, configured to receive and output a scattered echo signal reflected and/or scattered by a radiated body;

a microwave imaging unit in signal connection with the microwave receiving device and configured to receive the scattered echo signal, generate and determine a position, a shape, and a size of tumor tissue according to a set algorithm, and output imaging coordinate information;

a microwave temperature-measuring unit in signal connection with the microwave receiving device and configured to receive the scattered echo signal, generate a thermal imaging data on the position of the tumor tissue and surrounding according to a set algorithm, and output temperature measurement information;

a hyperthermia solution generating unit configured to generate or store hyperthermia parameter information required for killing the tumor tissue with different shapes and sizes at different positions in a human body;

a hyperthermia solution executing unit in data connection with the microwave imaging unit, the microwave temperature-measuring unit, and the hyperthermia solution generating unit, and configured to generate hyperthermia solution execution information used for killing the tumor tissue based on the imaging coordinate information, the temperature measurement information, and the hyperthermia parameter information which are already collected; and

a hyperthermia control device in control connection with the microwave radiation device and configured to receive the hyperthermia solution execution information, and, responding to the hyperthermia solution execution information, output the control signal for controlling action of the microwave radiation device.

Further, the hyperthermia apparatus body includes:

a hyperthermia bed including a bed body and a bed body support for supporting the bed body;

a radiation head mounting frame configured to be circular as a whole and slidably disposed relative to the bed body of the hyperthermia bed as a whole; and

a first driving device in transmission connection with the hyperthermia bed and/or the radiation head mounting frame, in control connection with the hyperthermia control device, and configured to receive the control signal from the hyperthermia control device and drive the hyperthermia bed and/or the radiation head mounting frame to move;

in which the microwave radiation device includes a plurality of microwave radiators movably disposed along the radiation head mounting frame and connected with the microwave source and power adapters, and a microwave radiation direction is set toward the bed body.

Further, a slidably movable groove is disposed along a length direction of the radiation head mounting frame and is slidably snap connected with a plurality of slidably movable mounting blocks, in which the plurality of microwave radiators are movably disposed on the plurality of slidably movable mounting blocks, respectively; and

a second driving device is disposed on the slidably movable groove and/or slidably movable mounting blocks for driving the slidably movable mounting blocks to move along the slidably movable groove, and is in control connection with the hyperthermia control device for receiving the control signal from the hyperthermia control device so as to drive the slidably movable mounting blocks to move to set positions.

Further, first sliding rails are disposed at two sides or two opposite side walls of the bed body and provided with first sliding blocks therein; a second sliding rail is disposed along a length direction of a slidably moving channel and provided with a second sliding block therein;

the radiation head mounting frame includes a first annular section and a second annular section, in which the first annular section is arc-shaped and fixedly connected with the first sliding blocks at two ends, and the second annular section is arc-shaped and fixedly connected with the second sliding block; or

the radiation head mounting frame is circular, and the bed body passes through the radiation head mounting frame in parallel with an axial direction of the radiation head mounting frame, with a bed body mounting base provided between the bed body and the ground and provided with a third sliding rail by which the bed body is slidably disposed relative to the bed body mounting base;

the first driving device comprises a screw transmission assembly or a chain transmission assembly for driving the first sliding blocks and the second sliding block to synchronously reciprocate along the first sliding rails and the second sliding rail, respectively, or for driving the bed body to reciprocate along the third sliding rail.

Further, the microwave radiators are movably connected with the slidably movable mounting blocks, with automatic adjustment members and/or manual adjustment members for adjusting the microwave radiation directions of the microwave radiators provided between the microwave radiators and the slidably movable mounting blocks.

Further, the hyperthermia solution executing unit includes:

a microwave radiator angle generating module configured to receive the imaging coordinate information, calculate a position of the hyperthermia bed relative to the radiation head mounting frame and a deflection angle of individual microwave radiators, and output an angle deflection parameter; and

a microwave radiator power generating module configured to receive the hyperthermia parameter information and generate and output an output power parameter of individual microwave radiators and a wavelength parameter and a frequency parameter of the microwave;

the hyperthermia control device includes:

a temporary data storage unit in data connection with the microwave imaging unit, the microwave temperature-measuring unit, and the hyperthermia solution executing unit, and configured to receive and store the imaging coordinate information, the temperature measurement information, the angle deflection parameter, the output power parameter, the microwave wavelength parameter, and the microwave frequency parameter;

a controller in electrical connection with the first driving device, the second driving device, and the power adapter of individual microwave radiators, and configured to receive the above-mentioned parameters and generate control signals for controlling motion of the first driving device, motion of the second driving device, and output power of individual power adapters; and

a feedback control unit in signal connection with the microwave temperature-measuring unit and configured to receive the temperature measurement information and output an adjusting signal to the controller thereby performing feedback adjustment on the control signals.

Further, automatic adjustment members are disposed between the microwave radiators and the slidably movable mounting blocks, in control connection with the controller and configured to receive control signals output from the controller and, responding to control signals output from the controller, adjust the microwave radiation directions of the microwave radiators; and

a radiation path planning module in data connection with the microwave radiator angle generating module and the microwave imaging unit and configured to receive the imaging coordinate information, calculate and generate a moving path of a radiation center point focused by the microwaves within a set time period according to the shape and the size of the tumor tissue, generate an angle deflection parameter table of individual microwave radiator with time as a reference variable based on the moving path, and output the angle deflection parameter table to the temporary data storage unit and/or the controller.

Further, the system includes a hyperthermia solution optimizing unit including the following modules:

a hyperthermia data storage module in data connection with the temporary data storage unit and configured to receive and store the imaging coordinate information, the temperature measurement information, the angle deflection parameter, the output power parameter, the wavelength parameter, and the frequency parameter corresponding to individual treatments; and

a hyperthermia effect analyzing module in data connection with the hyperthermia data storage module and configured to receive the imaging coordinate information of the tumor tissue after individual treatments, analyze and generate data on the change in the imaging coordinate information of the tumor tissue between individual treatments based on a built-in algorithm module, predict a growth trend of the tumor tissue and used hyperthermia parameter information, and generate a hyperthermia optimization solution which is stored or output to the hyperthermia solution generating unit.

Based on the control system of the physical targeted hyperthermia system used for tumor therapy, this application further provides a control method for the physical targeted hyperthermia system used for tumor therapy including:

taking a set plane or a set point in the hyperthermia apparatus body as a reference, calculating individual position coordinates in a three-dimensional space, and generating and storing information of individual position coordinates;

calculating, generating, and storing the hyperthermia parameter information table required for killing the tumor tissue with different shapes and different sizes at individual position coordinates;

scanning, obtaining, and temporarily storing shape information, size information, the position coordinate information, and current temperature information of the tumor tissue in the human body based on the microwave imaging unit and the microwave temperature-measuring unit;

selecting set hyperthermia parameters from the hyperthermia parameter information table based on the shape information, the size information, the position coordinate information, and the current temperature information of the tumor tissue;

generating corresponding hyperthermia solution execution information according to a set algorithm based on the set hyperthermia parameters; and

generating the corresponding control signals based on the hyperthermia solution execution information for controlling the action of the microwave radiation device and/or the hyperthermia apparatus body.

Further, calculating, generating, and storing the hyperthermia parameter information table required for killing the tumor tissue with different shapes and different sizes at individual position coordinates includes:

establishing a digital model of a human body and calculating a correlation between hyperthermia parameters and changes in temperatures of tissue at individual positions in the human body;

adding a digital model of tumor tissue with a set shape and a set size to the digital model of the human body, simulating microwave heating, adjusting the hyperthermia parameters, and obtaining simulation data; and

obtaining the hyperthermia parameters corresponding to target simulation data based on the simulation data, and generating and storing the hyperthermia parameter information table.

Compared with the prior art, this application has the following beneficial effects.

(1) By providing a plurality of microwave radiation probes on the radiation head mounting frame and using microwave imaging technology and microwave temperature measurement technology, the accurate positioning of the tumor tissue and focused heating can be achieved, thus tumor tissue and tumor cells can be killed without affecting normal human tissue, and non-invasive tumor therapy can be achieved.

(2) By carrying out real-time temperature measurement on the tumor tissue area during hyperthermia, the output power of individual microwave radiators and the position of the radiation center point can be adjusted according to changes in the temperature during hyperthermia, thereby achieving accurate heating on the tumor tissue.

(3) By comparing the shape and size of the tumor tissue after each therapy, the growth trend of the tumor tissue during the therapy is analyzed and generated, and microwave heating is performed in advance in the area where the tumor tissue may spread, thereby stopping the spread of the tumor tissue and improving the cure rate.

### BRIEF DESCRIPTION OF DRAWINGS

FIG. 1 is an overall schematic view of a physical targeted hyperthermia system used for tumor therapy according to an embodiment of this application;

FIG. 2 is an overall schematic view of the physical targeted hyperthermia system used for tumor therapy according to an embodiment of this application (omitting a bed body);

FIG. 3 is a partial enlarged schematic view of part A in FIG. 2;

FIG. 4 is an overall schematic view of a physical targeted hyperthermia system used for tumor therapy according to another embodiment of this application;

FIG. 5 is an overall schematic view of the physical targeted hyperthermia system used for tumor therapy according to another embodiment of this application (omitting a bed body mounting base);

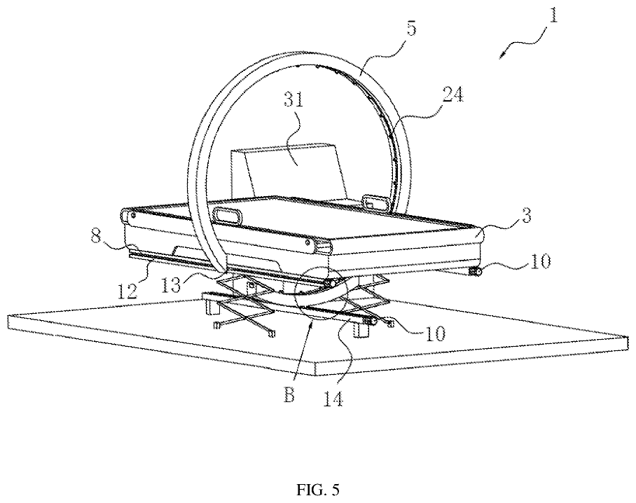

FIG. 6 is a partially enlarged schematic view of part B in FIG. 5;

FIG. 7 is a schematic framework view of one embodiment of this application; and

FIG. 8 is a schematic view of a control method for the physical targeted hyperthermia system used for tumor therapy according to one embodiment of this application.

In the drawings:
1. hyperthermia apparatus body
2. hyperthermia bed
3. bed body
4. bed body support
5. radiation head mounting frame
6. first annular section
7. second annular section
8. slidably movable groove
9. slidably movable mounting block
10. first driving device
11. bed body mounting base
12. first sliding rail
13. first sliding block
14. second sliding rail
15. second sliding block
16. microwave radiator angle generating module
17. microwave radiator power generating module
18. temporary data storage unit
19. controller
20. feedback control unit
21. radiation path planning module
22. hyperthermia data storage module
23. hyperthermia effect analyzing module
24. microwave radiator
25. microwave radiation device
26. microwave receiving device
27. microwave imaging unit
28. microwave temperature-measuring unit
29. hyperthermia solution generating unit
30. hyperthermia solution executing unit
31. hyperthermia control device
32. hyperthermia solution optimizing unit

### DETAILED DESCRIPTION

This application will be further explained in detail below in combination with embodiments and figures. These examples are only an explanation of this application, and do not limit the protection scope of this application.

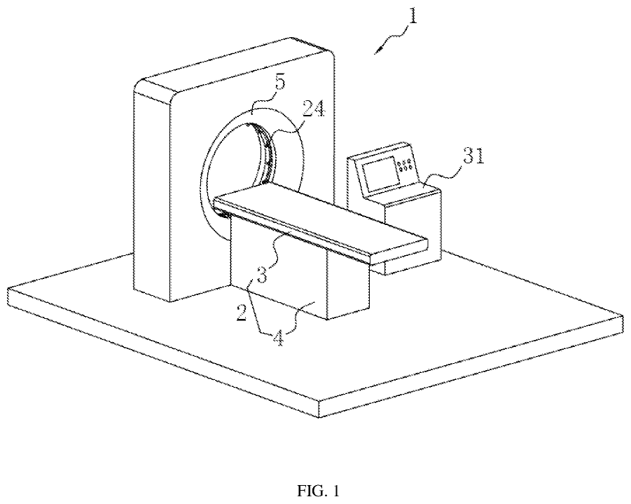

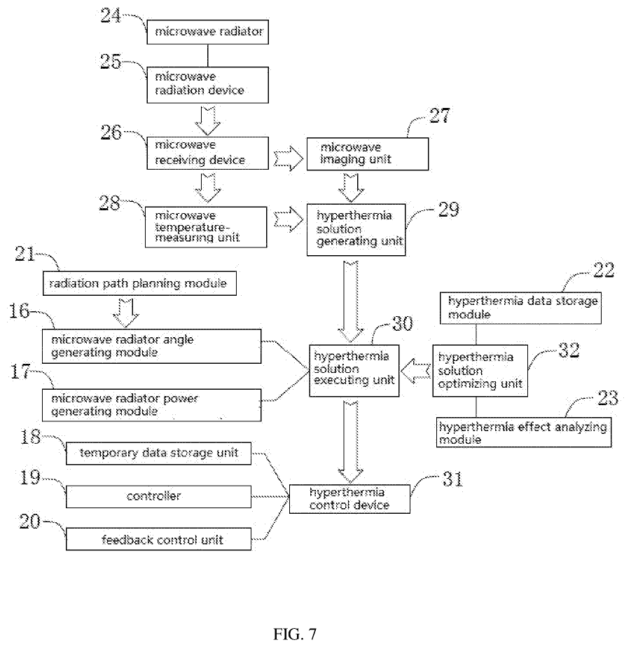

A physical targeted hyperthermia system used for tumor therapy, as shown in FIG. 1 and FIG. 7, mainly includes a hyperthermia apparatus body 1, a microwave radiation device 25, a microwave receiving device 26, a microwave imaging unit 27, a microwave temperature-measuring unit 28, a hyperthermia solution generating unit 29, a hyperthermia solution executing unit 30, and a hyperthermia control device 31.

The above-mentioned hyperthermia apparatus body 1 is used to provide at least one stable supporting surface for a patient and to provide mounting sites for individual function modules of the system during hyperthermia. The purpose of providing stable supporting surface is that, the position of the body of the patient can be relative stable with respect to the position of the microwave radiation device 25 when the patient is subject to hyperthermia, so that the position of a radiation center point focused by the microwaves can be fixed or stably moved along a set motion path.

Both the microwave radiation device 25 and the microwave receiving device 26 mentioned above are disposed on the hyperthermia apparatus body 1, and the microwave imaging unit 27, the microwave temperature-measuring unit 28, the hyperthermia solution generating unit 29, the hyperthermia solution executing unit 30, and the hyperthermia control device 31 are disposed separately from the above-mentioned hyperthermia apparatus body 1. In one embodiment, the microwave radiation device 25 and the microwave receiving device 26 may be in remote connection with the above-mentioned function units by the internet.

The microwave radiation device 25 is in connection with a microwave source, and is configured to generate a microwave with a set wavelength and a set frequency radiating toward a set direction according to a control signal from the hyperthermia control device 31. Specifically, in one embodiment, the above-mentioned microwave radiation device 25 includes a plurality of microwave radiators 24 connected with the microwave source and power adapters.

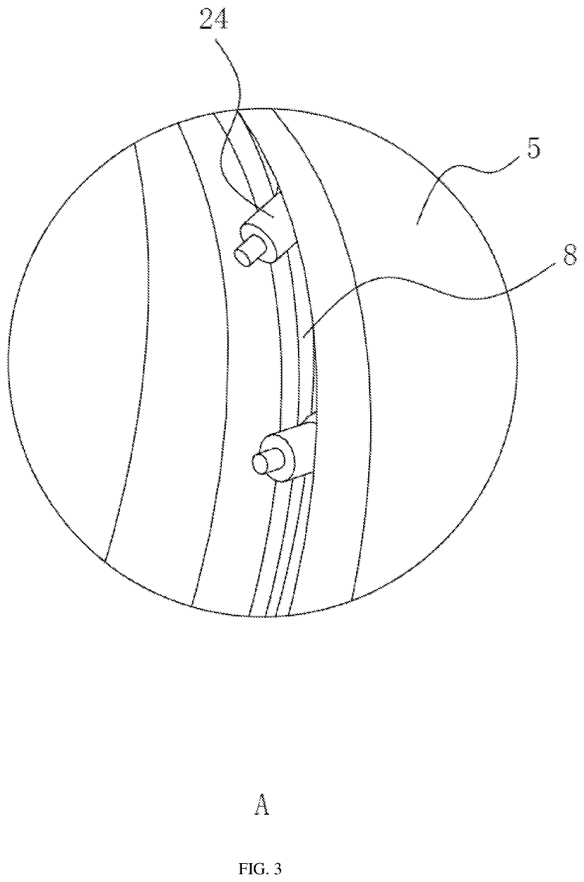

In order to enable a plurality of microwave radiators 24 mentioned above to generate microwaves stably radiating toward set directions, in one embodiment, as shown in FIG. 1 and FIG. 3, the hyperthermia apparatus body 1 includes: a hyperthermia bed 2, a radiation head mounting frame 5 slidably disposed relative to the hyperthermia bed 2, and a first driving device 10 used for driving the hyperthermia bed 2 to move relative to the radiation head mounting frame 5.

The hyperthermia bed 2 includes a bed body 3 and a bed body support 4 supporting the bed body 3. In one embodiment, a fork lifter may be adopted for the bed body support 4 in order to adjust the height of the hyperthermia bed 2. The struts of the fork lifter are respectively disposed at two sides of the bed body 3 in a width direction, so that a slidably moving channel is formed at the bottom of the bed body 3 and between two struts of the fork lifter.

The radiation head mounting frame 5 is circular as a whole and slidably disposed relative to the bed body 3 of the hyperthermia bed 2 as a whole. The first driving device 10, in transmission connection with the hyperthermia bed 2 and/or the radiation head mounting frame 5 and in control connection with the hyperthermia control device 31, is configured to receive the control signal from the hyperthermia control device 31 and to drive the hyperthermia bed 2 and/or the radiation head mounting frame 5 to move. A plurality of microwave radiators 24 are movably disposed along the radiation head mounting frame 5, and the microwave radiation is directed toward the bed body 3.

Specifically, the radiation head mounting frame 5 is provided with a slidably movable groove 8 along a length direction thereof and a plurality of slidably movable mounting blocks 9 slidably snap connected in the slidably movable groove 8, and a plurality of microwave radiators 24 are movably disposed in the plurality of slidably movable mounting blocks 9, respectively. A second driving device is disposed on the slidably movable groove 8 and/or slidably movable mounting blocks 9 for driving the slidably movable mounting blocks 9 to move along the slidably movable groove 8, and is in control connection with the hyperthermia control device 31 for receiving the control signal from the hyperthermia control device 31 so as to drive the slidably movable mounting blocks 9 to move to set positions. In one embodiment, a rack may be disposed at the length direction of the above-mentioned slidably movable groove 8, and a servo motor fixedly installed at the slidably movable mounting blocks 9 may be adopted as the second driving device, in which the end of the servo motor is fixedly provided with a gear engaged with the rack. The hyperthermia control device 31 is configured to control the movement of the above-mentioned slidably movable mounting blocks 9 by controlling the rotation of the servo motor.

In the above-mentioned technical solution, automatic adjustment of microwave radiation directions of a plurality of microwave radiators 24 mentioned above can be achieved by the hyperthermia control device 31, thereby achieving precise focusing of the microwaves and quick and accurate killing of tumor tissue in a human body.

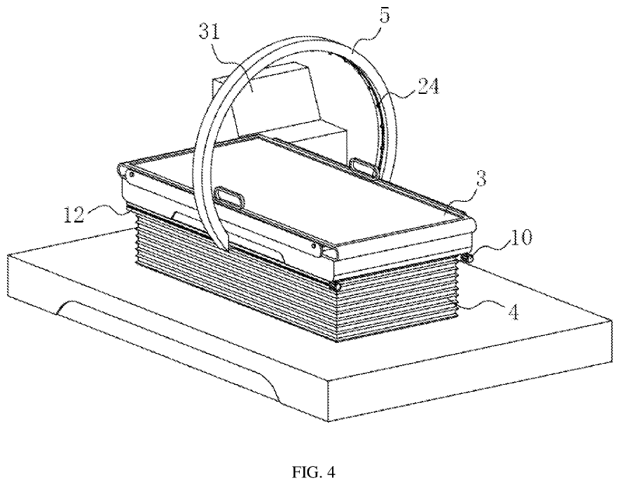

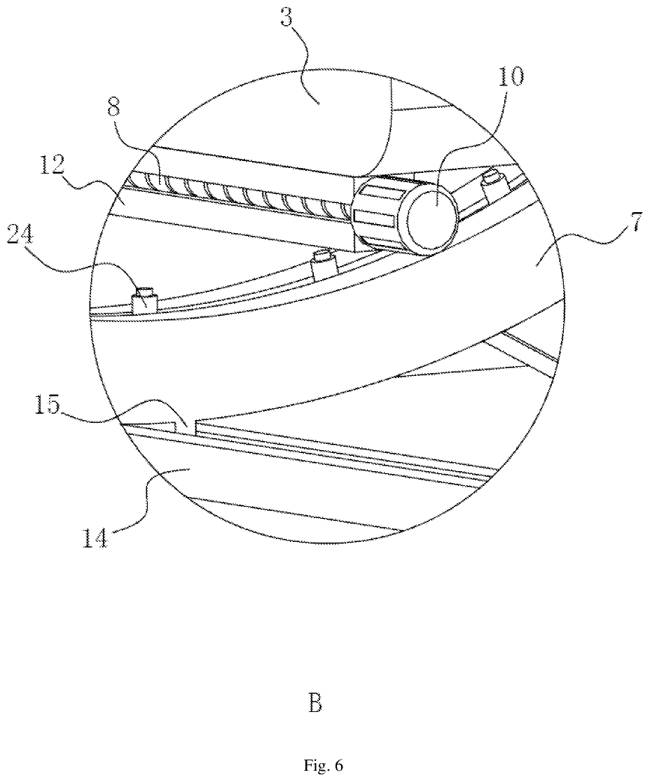

In one embodiment, as shown in FIG. 4 and FIG. 6, a first sliding rail 12 is disposed at two sides or two opposite side walls of the bed body 3, in which first sliding blocks 13 are slidably disposed, and a second sliding rail 14 is disposed along a length direction of the slidably moving channel, in which a second sliding block 15 is slidably disposed. The radiation head mounting frame 5 includes a first annular section 6 and a second annular section 7, in which the first annular section 6 is arc-shaped, and is fixedly connected with the first sliding blocks 13 at two ends, and the second annular section 7 is arc-shaped, and is fixedly connected with the second sliding block 15. The first annular section 6 and the second annular section 7 form an annulus, so that microwave radiation can be applied to any position of the human body from any direction without moving the bed body 3.

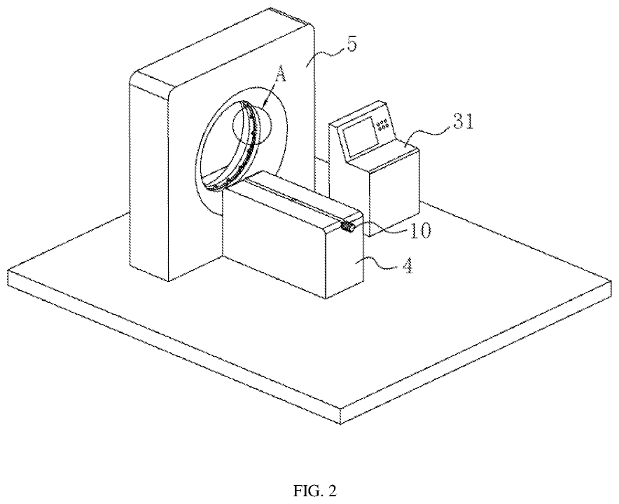

In another embodiment, as shown in FIG. 2 and FIG. 3, the radiation head mounting frame 5 is circular, and the bed body 3 passes through the radiation head mounting frame 5 in parallel to the axial direction thereof. There provided a bed body mounting base 11 between the bed body 3 and the ground, and the bed body mounting base 11 is provided with a third sliding rail by which the bed body 3 is slidably disposed relative to the bed body mounting base 11. Based on this configuration, microwave radiators 24 disposed on the radiation head mounting frame 5 can generate microwaves radiating the human body in all directions.

Preferably, the bed body 3 is made of a material that does not block the microwave beams, such as ceramics, glass, or a composition thereof.

The first driving device 10 includes a screw transmission assembly or a chain transmission assembly for driving the first sliding blocks 13 and the second sliding block to synchronously reciprocate along the first sliding rails 12 and the second sliding rail 14, respectively, or driving the bed body 3 to reciprocate along the third sliding rail. The first driving device 10 may be a servo motor in control connection with the control device, which achieves power output by driving the chain to move by a gear mounted on a motor shaft.

By adopting the above-mentioned technical solution, the radiation head mounting frame 5 is circular, so that microwave radiators 24 disposed thereon can generate microwaves radiating the tumor tissue in the human body in all directions, thereby achieving accurate heating on the tumor tissue, and reducing damage caused by the microwaves to normal tissue in human body as much as possible.

Specifically, the microwave radiators 24 are movably connected with the slidably movable mounting blocks 9, with automatic adjustment members and/or manual adjustment members for adjusting the microwave radiation directions of the microwave radiators 24 provided therebetween. In the above-mentioned technical solution, microwave radiation directions of the microwaves generated by the microwave radiators 24 may be adjusted by automatic control or manual control, thereby adjusting the radiation center point of the microwaves more accurately and achieving accurate heating on the tumor cells.

Preferably, automatic adjustment members are disposed between the microwave radiators 24 and the slidably movable mounting blocks 9, in control connection with the controller 19 and configured to receive and respond to control signals outputted by the controller 19 for adjusting the microwave radiation directions of the microwave radiators 24.

In one embodiment of this application, a microwave receiving device 26, such as a microwave receiver, is configured to receive and output a scattered echo signal reflected and/or scattered by a radiated body. It can be disposed in the same way as the microwave radiator 24, that is, movably disposed on the radiation head mounting frame 5, and can be changed regarding the position, so that it can receive the scattered echo signals reflected and/or scattered by the radiated body from different directions, convert them into an electrical signals via a set algorithm, and output them.

A microwave imaging unit 27 is in signal connection with the microwave receiving device 26, configured to receive the scattered echo signals, generate and determine a position, a shape, and a size of tumor tissue according to a set algorithm, and output imaging coordinate information. Different human tissues have different reflection shapes and changes in the temperature differ in different cells in human tissues when receiving microwave radiation with the same wavelength or frequency, by which characteristics it is easy to image tumor tissue in the human body and obtain the shape and size as well as a position coordinate of the tumor tissue.

A microwave temperature-measuring unit 28 is in signal connection with the microwave receiving device 26, configured to receive the scattered echo signal, generate a thermal imaging data on the position of the tumor tissue and surrounding according to a set algorithm, and output temperature measurement information. In practice, microwave is commonly used in temperature measurement, such as a microwave temperature measuring probe, etc., which will not be explained in detail hereafter.

A hyperthermia solution generating unit 29 is configured to generate or store hyperthermia parameter information required for killing the tumor tissue with different shapes and sizes at different positions in a human body. In practice, the hyperthermia solution generating unit 29 includes a computer or a specific data processor with a specific algorithm module built-in, being in data connection with the microwave imaging unit 27 and microwave temperature-measuring unit 28 via a local area network or remote internet. After inputting position coordinate information, shape information, and size information of the tumor tissue, corresponding hyperthermia parameter information can be quickly generated or be found in a data table, such as information on frequency, wavelength, radiation duration, and radiation direction of the microwave, and changes in the frequency or wavelength of the microwave during radiation.

A hyperthermia solution executing unit 30 is in data connection with the microwave imaging unit 27, microwave temperature-measuring unit 28, and hyperthermia solution generating unit 29, configured to generate information of executing the hyperthermia solution for killing the tumor tissue based on the collected imaging coordinate information, temperature measurement information, and hyperthermia parameter information. The information of executing the hyperthermia solution includes the angle at which the radiation head of each microwave radiator 24 should be deflected (that is, determining position parameters of individual microwave radiator 24 relative to the human body), driving parameters of the first driving device 10 and the second driving device, or the like.

A hyperthermia control device 31 is in control connection with the microwave radiation device 25, configured to receive the information of executing the hyperthermia solution, and, responding to the received information of executing the hyperthermia solution, output a control signal for controlling the action of the microwave radiation device 25, including changing the position and angle of the microwave radiators 24 or bed body 3, as well as the radiation power.

Further, as shown in FIG. 7, the hyperthermia solution executing unit 30 includes a microwave radiator angle generating module 16 and a microwave radiator power generating module 17.

The microwave radiator angle generating module 16 is configured to receive the imaging coordinate information, calculate the position of the hyperthermia bed 2 relative to the radiation head mounting frame 5 and a deflection angle of each microwave radiator 24, and output an angle deflection parameter and so on. The microwave radiator power generating module 17 is configured to receive the hyperthermia parameter information, and generate and output an output power parameter of individual microwave radiators 24 and a wavelength parameter and a frequency parameter of the microwave.

A radiation path planning module 21 is in data connection with the microwave radiator angle generating module 16 and the microwave imaging unit 27, configured to receive the imaging coordinate information, calculate and generate a moving path of the radiation center point focused by individual microwave radiators 24 within a set time period according to the shape and the size of the tumor tissue, generate an angle deflection parameter table of individual microwave radiators 24 with time as a reference variable based on the moving path, and output the angle deflection parameter table to a temporary data storage unit 18 and/or a controller 19. In the above technical solution, the microwave radiation directions of individual microwave radiator 24 can be automatically controlled by the controller 19, thereby changing the focusing point of individual microwave beams. By providing the radiation path planning module 21, the focusing point of individual microwave beams, that is, the radiation center point can be reasonably planned according to the shape and size of the tumor tissue, so that the moving path of the radiation center point covers the whole tumor tissue without damaging normal tissue cells, achieving more complete killing of the tumor tissue.

Further, the hyperthermia control device 31 includes the temporary data storage unit 18, the controller 19, and a feedback control unit 20. The temporary data storage unit 18 is in data connection with the microwave imaging unit 27, the microwave temperature-measuring unit 28, and the hyperthermia solution executing unit 30, configured to receive and store the imaging coordinate information, the temperature measurement information, the angle deflection parameter, the output power parameter, the microwave wavelength parameter, and the microwave frequency parameter. The controller 19 is in electrical connection with the first driving device 10, the second driving device, and the power adapter of each microwave radiator 24, configured to receive the above-mentioned parameters and generate control signals for controlling motion of the first driving device 10, motion of the second driving device, and output power of individual power adapters. In one embodiment, the hyperthermia control device 31 includes a single-chip computer module, an FPGA module, or a specific control circuit module which are in control connection with individual driving devices, microwave radiation device 25, and other function devices. The feedback control unit 20 is in signal connection with the microwave temperature-measuring unit 28, configured to receive the temperature measurement information and output an adjusting signal to the controller 19, thereby performing feedback adjustment on the control signals.

In the above-mentioned technical solution, the hyperthermia solution executing unit 30 outputs the angle deflection parameter, the output power parameter of individual microwave radiators 24, the wavelength parameter and frequency parameter of the microwave based on the hyperthermia parameter information output from the hyperthermia solution generating unit 29, and the controller 19 control the action of each microwave radiator 24, so that microwaves generated by microwave radiators 24 can be focused on a set position, thereby achieving accurate killing of cells of tumor tissue. In the above-mentioned process, the temporary data storage unit 18 can store the hyperthermia parameter information to be implemented so as to provide data support for a subsequent therapy, and the above-mentioned data can also be stored as historical data of the patient during therapy, which facilitates analyzing and re-examining the therapy process in a later time so as to obtain an optimized hyperthermia solution. By providing the feedback control unit 20, changes in temperature of the tumor tissue in the microwave focusing area during the whole therapy can be monitored in real time by the controller 19, and hyperthermia parameters can be adjusted in time according to the changes mentioned above, thereby achieving a closed-loop control for the hyperthermia, avoiding the temperature of the hyperthermia area from becoming too high or too low, for ensuring the effect of the hyperthermia.

For the purpose of improving the effect of the hyperthermia, the system is provided with a hyperthermia solution optimizing unit 32, including a hyperthermia data storage module 22 and a hyperthermia effect analyzing module 23. The hyperthermia data storage module 22 is in data connection with the temporary data storage unit 18, configured to receive and store the imaging coordinate information, temperature measurement information, angle deflection parameter, output power parameter, microwave wavelength parameter, and microwave frequency parameter corresponding to individual hyperthermia. The hyperthermia effect analyzing module 23 is in data connection with the hyperthermia data storage module 22, configured to receive the imaging coordinate information of the tumor tissue after individual hyperthermia, analyze and generate data on the change in the imaging coordinate information of the tumor tissue between individual hyperthermia based on a built-in algorithm module, including the difference in the shape and size of the tumor tissue between individual hyperthermia, predict a growth trend of the tumor tissue and used hyperthermia parameter information, and generate a hyperthermia optimization solution which is stored or output to the hyperthermia solution generating unit 29. For example, microwave heating is performed in advance in the area into which the tumor tissue may spread, thereby stopping the spread of the tumor tissue.

The present application may have the working principle and beneficial effects as follow.

When the microwave is used to kill tumor cells or tumor tissue in the human body, the microwave radiation device 25 firstly radiates microwave with a set wavelength and frequency generated toward a human body, the microwave receiving device 26 receives the scattered echo signals reflected and/or scattered by the human body, and the microwave imaging unit 27 generates and determines the specific position of the tumor tissue in the human body by using the above-mentioned scattered echo signals, thereby outputting the imaging coordinate information; and the hyperthermia solution generating unit 29 generates corresponding hyperthermia parameter information based on the above-mentioned imaging coordinate information, and then the hyperthermia solution executing unit 30 generates a corresponding therapy solution based on the above-mentioned hyperthermia parameter information. The hyperthermia control device 31 accurately controls the microwave radiation device 25 to perform continuous microwave heating at a certain position of the human body according to the above-mentioned hyperthermia solution execution information, thereby eliminating tumor cells or tumor tissue at the lesion.

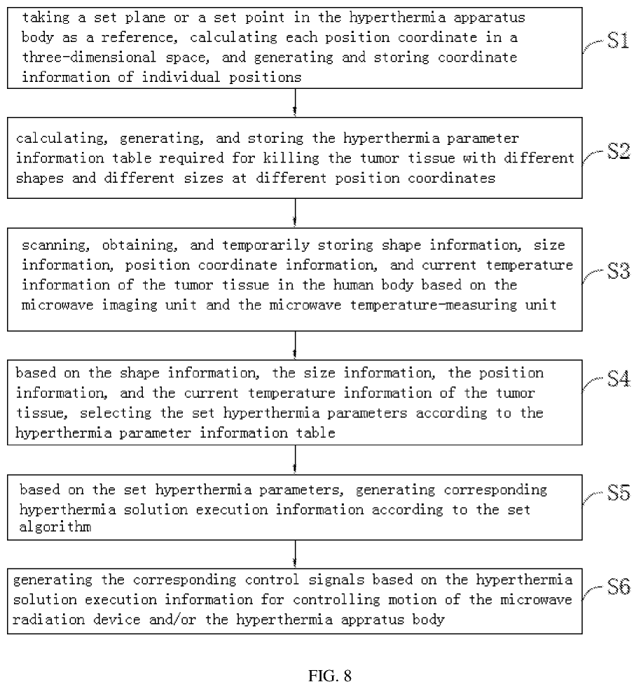

Based on the control system of the physical targeted hyperthermia system used for tumor therapy, this application also provides a control method for the physical targeted hyperthermia system used for tumor therapy, as shown in FIG. 8, including the following steps:

S1, taking a set plane or a set point in the hyperthermia apparatus body 1 as a reference, calculating each position coordinate in a three-dimensional space, and generating and storing coordinate information of individual positions;

S2, calculating, generating, and storing the hyperthermia parameter information table required for killing the tumor tissue with different shapes and different sizes at different position coordinates;

S3, scanning, obtaining, and temporarily storing shape information, size information, the position coordinate information, and current temperature information of the tumor tissue in the human body based on the microwave imaging unit 27 and the microwave temperature-measuring unit 28;

S4, selecting the set hyperthermia parameters according to the hyperthermia parameter information table based on the shape information, the size information, the position coordinate information, and the current temperature information of the tumor tissue;

S5, generating corresponding hyperthermia solution execution information according to the set algorithm based on the set hyperthermia parameters; and

S6, generating the corresponding control signals based on the hyperthermia solution execution information for controlling motion of the microwave radiation device 25 and/or the hyperthermia apparatus body 1.

Further, S2 includes:

S21, establishing a human body digital model and calculating a correlation between hyperthermia parameters and changes in temperatures of tissue at different positions in the human body;

S22, adding a digital model of tumor tissue with a set shape and a set size to the human body data model, simulating microwave heating, adjusting the hyperthermia parameters, and obtaining simulation data; and

S23, obtaining the hyperthermia parameters corresponding to target simulation data based on the simulation data, and generating and storing the hyperthermia parameter information table.

The above-mentioned S6 also includes providing real time feedback of changes in the temperature of the tumor tissue area during the hyperthermia, predicting the trend of changes of the temperature, and adjusting the hyperthermia parameters when the above-mentioned temperature reaches or is about to reach a set value, thereby achieving closed-loop dynamic control.

It should be noted that in S4, the above-mentioned hyperthermia parameters include, but not limited to, frequency, wavelength, and radiation direction of each microwave, the motion path of the radiation center point, and other parameters.

The embodiments described in the detailed description are all preferred embodiments of this application, and should not be considered as limit the scope of this application thereto. Therefore, all the equivalent changes made to the structure, shape and principle of this application should be encompassed within the protection scope of this application.

What is claimed is:

1. A physical targeted hyperthermia system used for tumor therapy, comprising:
a hyperthermia apparatus body, configured to provide at least one stable supporting surface for a patient during hyperthermia and provide mounting positions for individual system function modules;
a microwave radiation device, in connection with a microwave source and configured to generate a microwave with a set wavelength and a set frequency radiating toward a set direction according to a control signal;
a microwave receiving device, configured to receive and output a scattered echo signal reflected and/or scattered by a radiated body;
a microwave imaging unit, in signal connection with the microwave receiving device and configured to receive the scattered echo signal, generate and determine a position, a shape, and a size of tumor tissue according to a set algorithm, and output imaging coordinate information;
a microwave temperature-measuring unit, in signal connection with the microwave receiving device and configured to receive the scattered echo signal, generate a thermal imaging data on the position of the tumor tissue and surrounding according to a set algorithm, and output temperature measurement information;
a hyperthermia solution generating unit, configured to generate or store hyperthermia parameter information required for killing the tumor tissue with different shapes and sizes at different positions in a human body;
a hyperthermia solution executing unit, in data connection with the microwave imaging unit, the microwave temperature-measuring unit, and the hyperthermia solution generating unit, and configured to generate hyperthermia solution execution information used for killing the tumor tissue based on the imaging coordinate information, the temperature measurement information, and the hyperthermia parameter information which have been acquired; and
a hyperthermia control device, in control connection with the microwave radiation device and configured to receive the hyperthermia solution execution information, and, responding to the hyperthermia solution execution information, output the control signal for controlling action of the microwave radiation device.

2. The physical targeted hyperthermia system used for tumor therapy according to claim 1, the hyperthermia apparatus body comprising:
a hyperthermia bed comprising a bed body and a bed body support for supporting the bed body;
a radiation head mounting frame, configured to be circular as a whole and slidably disposed relative to the bed body of the hyperthermia bed wholly; and
a first driving device, in transmission connection with the hyperthermia bed and/or the radiation head mounting frame and in control connection with the hyperthermia control device, and configured to receive the control signal from the hyperthermia control device and drive the hyperthermia bed and/or the radiation head mounting frame to move;
wherein the microwave radiation device comprises a plurality of microwave radiators movably disposed along the radiation head mounting frame and connected with the microwave source and power adapters, and a microwave radiation direction is set toward the bed body.

3. The physical targeted hyperthermia system used for tumor therapy according to claim 2, wherein
a slidably movable groove is disposed along a length direction of the radiation head mounting frame and slidably snap connected with a plurality of slidably movable mounting blocks on which a plurality of microwave radiators are movably disposed, respectively; and
a second driving device is disposed on the slidably movable groove and/or slidably movable mounting blocks for driving the slidably movable mounting blocks to move along the slidably movable groove, and is in control connection with the hyperthermia control device for receiving the control signal from the hyperthermia control device so as to drive the slidably movable mounting blocks to move to set positions.

4. The physical targeted hyperthermia system used for tumor therapy according to claim 3, wherein
a first sliding rail is disposed at two sides or two opposite side walls of the bed body, and provided with a first sliding block therein; a second sliding rail is disposed along a length direction of a slidably moving channel and provided with a second sliding block therein; the radiation head mounting frame comprises a first annular section and a second annular section, in which the first annular section is arc-shaped and fixedly connected with the first sliding blocks at two ends, and the second annular section is arc-shaped and fixedly connected with the second sliding block; or
the radiation head mounting frame is configured to be circular, and the bed body passes through the radiation head mounting frame in parallel with an axial direction of the radiation head mounting frame, with a bed body mounting base provided between the bed body and the ground and provided with a third sliding rail by which the bed body is slidably disposed relative to the bed body mounting base;
the first driving device comprises a screw transmission assembly or a chain transmission assembly for driving the first sliding blocks and the second sliding block to synchronously reciprocate along the first sliding rails and the second sliding rail, respectively, or for driving the bed body to reciprocate along the third sliding rail.

5. The physical targeted hyperthermia system used for tumor therapy according to claim 3, wherein the microwave radiators are movably connected with the slidably movable mounting blocks, and automatic adjustment members and/or manual adjustment members for adjusting the microwave radiation directions of the microwave radiators are provided between the microwave radiators and the slidably movable mounting blocks.

6. The physical targeted hyperthermia system used for tumor therapy according to claim 5, wherein
the hyperthermia solution executing unit comprises:
a microwave radiator angle generating module, configured to receive the imaging coordinate information, calculate a position of the hyperthermia bed relative to the radiation head mounting frame and a deflection angle of individual microwave radiators, and output an angle deflection parameter; and
a microwave radiator power generating module, configured to receive the hyperthermia parameter information and generate and output an output power parameter of individual microwave radiators and a microwave wavelength parameter and a microwave frequency parameter;
the hyperthermia control device comprises:
a temporary data storage unit, in data connection with the microwave imaging unit, the microwave temperature-measuring unit, and the hyperthermia solution executing unit, and configured to receive and store the imaging coordinate information, the temperature measurement information, the angle deflection parameter, the output power parameter, the microwave wavelength parameter, and the microwave frequency parameter;
a controller, in electrical connection with the first driving device, the second driving device, and the power adapters of individual microwave radiators, and configured to receive the above-mentioned parameters and generate control signals for controlling motion of the first driving device, motion of the second driving device, and output power of individual power adapters; and
a feedback control unit, in signal connection with the microwave temperature-measuring unit and configured to receive the temperature measurement information and output an adjusting signal to the controller for performing feedback adjustment to the control signals.

7. The physical targeted hyperthermia system used for tumor therapy according to claim 6, wherein
automatic adjustment members are disposed between the microwave radiators and the slidably movable mounting blocks, in control connection with the controller, and are configured to receive control signals outputted by the controller, and, responding to the control signals outputted by the controller, adjust the microwave radiation directions of the microwave radiators; and
a radiation path planning module is in data connection with the microwave radiator angle generating module and the microwave imaging unit, and configured to receive the imaging coordinate information, calculate and generate a moving path of a radiation center point focused by the microwaves within a set time period according to the shape and the size of the tumor tissue, generate an angle deflection parameter table of individual microwave radiators with time as a reference variable based on the motion path, and output the angle deflection parameter table to the temporary data storage unit and/or the controller.

8. The physical targeted hyperthermia system used for tumor therapy according to claim 6, wherein the system further comprises a hyperthermia solution optimizing unit comprising:
a hyperthermia data storage module, in data connection with the temporary data storage unit, and configured to receive and store the imaging coordinate information, the temperature measurement information, the angle deflection parameter, the output power parameter, the wavelength parameter, and the frequency parameter corresponding to individual treatments; and
a hyperthermia effect analyzing module, in data connection with the hyperthermia data storage module, and configured to receive the imaging coordinate information of the tumor tissue after individual treatments, analyze and generate data on the change in the imaging coordinate information of the tumor tissue between individual treatments based on a built-in algorithm module, predict a growth trend of the tumor tissue and used hyperthermia parameter information, and generate a hyperthermia optimizing solution which is stored or output to the hyperthermia solution generating unit.

9. A control method for the physical targeted hyperthermia system used for tumor therapy based on the physical targeted hyperthermia system used for tumor therapy according to claim 8, comprising:
taking a set plane or a set point in the hyperthermia apparatus body as a reference, calculating individual position coordinates in a three-dimensional space, and generating and storing information of individual position coordinates;
calculating, generating, and storing the hyperthermia parameter information table required for killing the tumor tissue with different shapes and different sizes at individual position coordinates;
scanning, obtaining, and temporarily storing shape information, size information, the information of position coordinates, and current temperature information of the tumor tissue in the human body based on the microwave imaging unit and the microwave temperature-measuring unit;
selecting set hyperthermia parameters from the hyperthermia parameter information table based on the shape information, the size information, the information of position coordinates, and the current temperature information of the tumor tissue;
generating corresponding hyperthermia solution execution information according to a set algorithm based on the set hyperthermia parameters; and
generating the corresponding control signals based on the hyperthermia solution execution information for controlling action of the microwave radiation device and/or the hyperthermia apparatus body.

10. The control method according to claim 9, wherein calculating, generating, and storing the hyperthermia parameter information table required for killing the tumor tissue with different shapes and different sizes at individual position coordinates comprises:
establishing a digital model of a human body and calculating a correlation between hyperthermia parameters and changes in temperatures of tissue at different positions in the human body;
adding a digital model of tumor tissue with a set shape and a set size to the digital model of the human body, simulating microwave heating, adjusting the hyperthermia parameters, and obtaining simulation data; and
obtaining the hyperthermia parameters corresponding to target simulation data based on the simulation data, and generating and storing the hyperthermia parameter information table.
